(19) United States
(12) Patent Application Publication
(10) Pub. No.: US 2018/0333350 A1
(43) Pub. Date: Nov. 22, 2018

(54) LIQUID LOTION (CREAM FOR SKIN) FOR MOISTURIZING AND CELLS RENEWAL APPLIED TO FACE, BODY AND HANDS

(71) Applicants: Dimitrios ANASTASOPOULOS, Athens (GR); Mahi-Stefania VRONTOU, Athens (GR); Alkaterini ANASTASOPOULOU, Athens (GB); Vassiliki ANASTASOPOULOU, Athens (GB)

(72) Inventor: Dimitrios ANASTASOPOULOS, Athens (GR)

(21) Appl. No.: 15/767,654

(22) PCT Filed: Oct. 12, 2016
(86) PCT No.: PCT/GR2016/000051

§ 371 Date: Apr. 12, 2018

(30) Foreign Application Priority Data
Oct. 12, 2015 (GR) 20150100441

(51) Int. Cl. A61K 8/9789; A61Q 19/00; A61Q 19/08; A61Q 19/06; A61K 8/96; A61K 8/67; A61K 8/9794; A61K 8/34; A61K 8/65; A61K 8/73

(52) U.S. Cl. CPC A61K 8/9789; A61Q 19/007; A61Q 19/08; A61Q 19/06; A61K 8/96; A61K 8/671; A61K 2800/5922; A61K 8/678; A61K 8/9794; A61K 8/345; A61K 8/65; A61K 8/735; A61K 8/676

(57) ABSTRACT

Cosmetic use product for moisturizing/cells renewal which consists of a mixture of plant origin's ingredients in liquid form, as they were presented in the part of “declarations”. These particular ingredients are exposed to a procedure of warming up through a heating machine appropriate for this use. The advantage of this product is that since its ingredients are in liquid form and they have undergone previously heating, they penetrate more in depth and skin is being hydrated in a larger extent.

This invention is related to a product for cosmetic use of plant origin, which has liquid form and is exposed to heating.

There are plenty and well -known products for cosmetic use, which are used for face and body moisturizing and for cells renewal.

Therefore, this product is in liquid form and it can be warmed up through a heating machine, that it can be found in trade, in order that its ingredients could penetrate into the deepest layers of body and skin. The skin would remain also moisturized and the optimal renewal of the skin's cells could be achieved for a longer period of time. More precisely, this particular beauty product consists of the following ingredients:
Liquid silk (0.01%-3%)
Vitamin A (0.001%-1%)
Vitamin C (0.001-0,5%)
Vitamin E (0.001-2%)
Rose water (10%-80%)
Aloe flower water (10%-80%)
Vegetable Glycerin (0.01%-5%)
Preservative (phenoexyethanol ethylexyglycerin) (0.01%-1%)
Collagen (0.001%-1%)
Hyaluronic Acid (0.001%-1%)

Vitamins A, C and E contribute respectively to cells differentiation, they try to reduce skin aging. They also contribute to the smoothing of the wrinkles and the protection of the extreme skin layers from the damage caused by the UV radiation.

Liquid silk combined with these three vitamins gives this product powerful anti-aging properties. Rose water stimulates, tightens skin and prevents the creation of wrinkles. Aloe gel, which is anti-inflammatory, combined with aloe flower water acts against wrinkles and increases skin collagen.

Taking of liquid collagen increases the density of collagen fibrils and fibroblasts and as a result the look of skin is improving.

Hyaluronic acid is extremely important for maintaining skin's moisturizing and elastic properties against deleterious external factors and enforces skin's healing reconstructive property.

The preservative (phenoexyethanol ethylexyglycerin) contributes to the maintenance of the product for the period of one year and doesn't cause irritations to the skin.

The advantage of this invention is that the optimal moisturizing of face and body is achieved. Cells are also being renewed in more depth, than the common products for cosmetic use, since the product has liquid form and as it is heated, its ingredients are becoming more efficient and effective. It is suitable for the whole of skin (face and body) and acts especially in considerably different parts of face and body.

According with the invention, this moisturizing/restauration product could be enriched with other ingredients (essences and mild chemicals). A particular attention has to be given to the combination of the quantities used so that allergies to the skin won't appear, cells differentiation will be achieved and the efficiency of the ingredients in use will be enforced.

1. The cosmetic use product for moisturizing/cells renewal is formed by a mixture in liquid form, which consists of the following ingredients:
Liquid silk (0.01%-3%)
Vitamin A (0.001%-1%)
Vitamin C (0.001-0,5%)
Vitamin E (0.001-2%)
Rose water (10%-80%)
Aloe flower water (10%-80%)
Vegetable Glycerin (0.01%-5%)
Preservative (phenoexyethanol ethylexyglycerin) (0.01%-1%)
Collagen (0.001%-1%)
Hyaluronic Acid (0.001%-1%)
The cosmetic use product for moisturizing/cells renewal, according with this invention, has the characteristic that it consists of a mixture of plant's origin ingredients. These ingredients are in liquid form and are exposed to heating. A simple way of their warming up is through a heating machine, that it can be found in trade.
Consequently, the product becomes more efficient, skin's moisturizing has more duration and penetrates more deeply into the skin's pores. Also, the cells are being renewed in greater extent and more precisely in areas of the body particularly sensitive, such as those affected by cellulite, stretch marks and wrinkles.
